(19) United States
(12) Patent Application Publication
(10) Pub. No.: US 2022/0403319 A1
(43) Pub. Date: Dec. 22, 2022

(54) Methods for Isolating Single-Molecule Products

(71) Applicant: Locus IP Company, LLC, Solon, OH (US)

(72) Inventors: Sean FARMER, Ft. Lauderdale, FL (US); Ken ALIBEK, Solon, OH (US)

(21) Appl. No.: 17/765,548

(22) PCT Filed: Feb. 4, 2021
(86) PCT No.: PCT/US2021/016521

§ 371 Date: Mar. 31, 2022

Related U.S. Application Data
(60) Provisional application No. 62/983,621, filed on Feb. 29, 2020.

(51) Int. Cl. C12N 1/06; C12N 1/16; C12P 19/04; C12P 19/44; C12P 19/62; C12P 7/6436

(52) U.S. Cl. CPC C12N 1/063; C12N 1/16; C12P 19/04; C12P 19/445; C12P 19/62; C12P 7/6436

(57) ABSTRACT

The subject invention provides materials and methods for producing, isolating, extracting and purifying single-molecule products. The subject invention provides materials and methods for extracting microbial metabolites at a high level of purity, for example, a purity of at least 80% by weight, and preferably at least 95% by weight or more. Specifically, the subject invention provides materials and methods for isolating or extracting biosurfactants and polyketides at a high level of purity. Preferably, the biosurfactant is a sophorolipid (SLP).

### CROSS-REFERENCE TO RELATED APPLICATION

This application claims priority to U.S. Provisional Patent Application No. 62/983,621, filed Feb. 29, 2020, which is incorporated by reference herein in its entirety.

### BACKGROUND OF THE INVENTION

Microbial metabolites are, in general, small molecular compounds produced by microorganisms, including primary metabolites such as saccharides, amino acids, and lipids, and secondary metabolites such as biosurfactants, polyketides, alkaloids, flavonoids, glycosides, quinolones, terpenoids, peptides and growth factors. Primary microbial metabolites are essential compounds for the survival of microorganisms while secondary microbial metabolites usually are compounds having important bioactive functions that can be used in a wide range of applications in medicine and agriculture. For example, many secondary microbial metabolites are used as antitumor, antiviral and antibacterial drugs.

Polyketides are a large group of compounds that contain carbonyl and methylene groups. Polyketides are commonly used in the pharmaceutical industry as antibiotics (e.g., erythromycin, clarithromycin, azithromycin, nystatin, avermectin, spiramycin and tetracyclines), as immunosuppressants (e.g., tacrolimus, rapamycin, radicicol and pochonin), as hypocholesterolemic agents (e.g., lovastatin), as anti-cancer agents (e.g. doxorubicin) and as anthelmintic agents (e.g., albendazole, mebendazole, diethylcarbamazine, ivermectin, and praziquantel), as well as in agricultural pest management as insecticides (e.g., spinosyn A).

Erythromycin has been used for the treatment of a number of bacterial infections. There are four types of erythromycin: A, B, C, and D. These four types of Erythromycin have different antibacterial activities and different active concentrations. Erythromycin A, the major and most active type of erythromycin, is used in the pharmaceutical industry as an antibiotic and a precursor for other antibiotics such as azitromycin, clarithromycin and roxithromycin. However, when erythromycin A is produced by standard methods using the bacterium Saccharopolyspora erythraea, erythromycin B, C and D may compose up to 30% of the obtained yield. Thus, in order to achieve a marketable polyketide product, e.g., erythromycin A, methods for extracting and purifying such compounds from microorganisms are desired.

Biosurfactants are a structurally diverse group of surface-active substances produced by microorganisms. All biosurfactants are amphiphiles. They have two parts: a polar (hydrophilic) moiety and a non-polar (hydrophobic) group. Due to their amphiphilic structure, biosurfactants can, for example, increase the surface area of hydrophobic water-insoluble substances, increase the water bioavailability of such substances, and change the properties of bacterial cell surfaces.

Biosurfactants can also reduce the interfacial tension between water and oil and, therefore, lower the hydrostatic pressure required to move entrapped liquid to overcome the capillary effect. Biosurfactants accumulate at interfaces, thus reducing interfacial tension and leading to the formation of aggregated micellar structures in solution. The formation of micelles provides a physical mechanism to mobilize, for example, oil in a moving aqueous phase. The ability of biosurfactants to form pores and destabilize biological membranes also permits their use as antibacterial, antifungal, and hemolytic agents to, for example, control pest and/or microbial growth.

Like chemical surfactants, the properties of biosurfactants can be measured by hydrophile-lipophile balance (HLB). HLB is the balance of the size and strength of the hydrophilic and lipophilic moieties of a surface-active molecule. Specific HLB values are required for a stable emulsion to be formed. In water/oil and oil/water emulsions, the polar moiety of the surface-active molecule orients towards the water, and the non-polar group orients towards the oil, thus lowering the interfacial tension between the oil and water phases.

There are multiple types of biosurfactants, including glycolipids, lipopeptides, flavolipids phospholipids, fatty acid esters, and high molecular weight polymers such as lipoproteins, lipopolysaccharide-protein complexes, and polysaccharide-protein-fatty acid complexes. These biosurfactants can be used in various industries such as the petroleum industry, agriculture, cosmetics and pharmaceuticals.

Glycolipids, in particular, are biosurfactants comprising a carbohydrate and at least one fatty acid. Glycolipids include, for example, rhamnolipids (RLP), rhamnose-d-phospholipids, trehalose lipids, trehalose dimycolates, trehalose monomycolates, mannosylerythritol lipids (MEL), cellobiose lipids, ustilagic acids and/or sophorolipids (SLP).

Sophorolipids are glycolipids that comprise a sophorose consisting of two glucose molecules, linked to a fatty acid by a glycosidic ether bond. They are categorized into two general forms: the lactonic form, where the carboxyl group in the fatty acid side chain and the sophorose moiety form a cyclic ester bond; and the acidic form, or linear form, where the ester bond is hydrolyzed. In addition to these forms, there exists a number of derivatives characterized by the presence or absence of double bonds in the fatty acid side chain, the length of the carbon chain, the position of the glycosidic ether bond, the presence or absence of acetyl groups introduced to the hydroxyl groups of the sugar moiety, and other structural parameters.

Lactonic and acidic sophorolipids have different functional properties. For example, acidic SLP have higher HLB than lactonic SLP, while lactonic SLP have lower HLB and greater surface tension reducing properties than acidic SLP. Additionally, acidic SLP are highly water soluble due to their free carboxylic acid groups. Combining lactonic and acidic SLP in different ratios affects, e.g., emulsion droplet size, viscosity reducing properties, and surface/interfacial tension reduction properties.

SLP can be used in, for example, food preservation, biomedicine, cosmetics, bioremediation, remediation of heavy metals, and making various household cleaning products. SLP can also be applicable to the petroleum industry in, for example, drilling, cement slurries, fracturing, enhanced oil recovery, scale formation prevention, acidization, demulsification of crude fluids, corrosion inhibition, reduced oil viscosity, cleaning of equipment, waterflooding, and/or foam and steam flooding. Furthermore, in agriculture and livestock production, SLP can be used as, for example, soil amendments, broad spectrum biopesticides, antiviral, antifungal and antibacterial agents, and/or additives to animal feed to enhance nutrient absorption.

Fermentation of yeast cells in a culture substrate including a sugar and/or lipids and fatty acids with carbon chains of differing length can be used to produce a variety of sophorolipids. The yeast Starmerella bombicola is one of the most widely recognized producers of SLP. Typically, the yeast produces both lactonic and acidic SLP during fermentation, with about 60-70% of the SLP comprising lactonic forms, and the remainder comprising acidic forms. Thus, the standard SLP product produced using current production methods can only be used in narrow applications because the range of, e.g., HLB value, is also narrow, e.g., between 4 and 9.

Additionally, because of the nature of biological processes, it is difficult to standardize the exact concentration of pure SLP that can be extracted from a yeast culture medium. Furthermore, crude form SLP can have a cloudy appearance and an undesirable smell. Thus, in order to obtain a desired concentration and appearance and/or smell for a marketable SLP product, it is often necessary to purify the SLP. However, obtaining highly purified forms of SLP (e.g., greater than 95%), particularly the acidic form, from a cultivation batch is challenging and costly.

The traditional method for extraction and purification of erythromycin A involves the isolation of erythromycin-isothiocyanate, preparation of erythromycin base and separation and purification of erythromycin A. For example, the whole broth needs to be adjusted to an alkaline pH, e.g., 8.5-9.5. After removing the biomass by centrifugation, the supernatant is filtered and mixed with methylisobutylketone for phase separation by centrifugation. Once the organic phase is collected and heated to 30° C., Na isothiocyanate solution is added and the mixture is adjusted to a pH of 5.8. Erythromycin-isothiocyanate crystalizes by cooling to 10° C. with mild agitation. Erythromycin-isothiocyanate is then isolated after filtering, washing and drying process.

To prepare the erythromycin base, erythromycin-isothiocyanate is re-suspended in dichloromethane under mild stirring at 33° C. with the addition of NaOH solution to adjust the pH to between 9.5 and 10.0. Such mixture is added with filter aid (Perlite) and activated carbon, and then filtered. After wash with a small quantity of dichloromethane, the organic phase is extracted with deionized water at 33° C. under slow stirring. After phase separation, the aqueous phase is discarded and the organic phase with an increased concentration of erythromycin induces the precipitation of the macrolide. The precipitated erythromycin can be recovered by centrifugation or filtration, followed by the steps of washing with dichloromethane, and drying at 50° C. The obtained crude erythromycin base is re-suspend in deionized water, heated to 50-60° C., and mixed with lauryl sulfate. Purified erythromycin base can be obtained by centrifugation or filtration followed by washing with deinonized water at 60° C. Erythromycin A can then be separated and purified from impure EM with macroporous resin SP825 by 2-stepwise elution chromatography.

In order to obtain highly purified chemical substances including polyketides and biosurfactants, it is necessary to use several methods including initial recovery, purification with the use of sophisticated equipment and methodology which make the process labor extensive, expensive and with limited ability to obtain highly pure single-molecule product.

Therefore, there is a need for safe, cost-effective and environmentally-friendly methods for extracting small-molecular compounds, such as polyketides and biosurfactants, with a high level of purity, and optionally, suitable for industrial scale applications.

### BRIEF SUMMARY OF THE INVENTION

The subject invention provides materials and methods for producing, isolating, extracting and purifying microbial metabolites at a high level of purity. Specifically, the subject invention provides materials and methods for the purification of microbial metabolites to a purity of, for example, at least 80% by weight, and preferably at least 95% by weight or more.

The subject invention also provides materials and methods for producing, extracting and purifying a single-molecule product that is substantially free of other compounds or cellular materials. The purified or isolated single-molecule product may have a purity of, for example, at least 80% by weight, at least 85% by weight, at least 90% by weight, at least 95% by weight, at least 98% by weight, or at least 99% by weight, at least 99.9% by weight or at least 100% by weight.

In one embodiment, the single-molecule product comprises a single microbial metabolite produced by microorganisms. In one embodiment, the microbial metabolite is selected from primary metabolites such as saccharides, amino acids, and lipids, and secondary metabolites such as biosurfactants, polyketides, fatty acid esters, alkaloids, flavonoids, glycosides, quinolones, terpenoids, peptides and growth factors. In a further embodiment, the microbial metabolite is selected from biosurfactants and polyketides.

In one embodiment, the biosurfactants include glycolipids, lipopeptides, flavolipids, phospholipids, fatty acid esters, and high molecular weight polymers such as lipoproteins, lipopolysaccharide-protein complexes, and polysaccharide-protein-fatty acid complexes. Preferably, the biosurfactants are glycolipids that comprise a carbohydrate and at least one fatty acid. Glycolipids include, for example, rhamnolipids (RLP), rhamnose-d-phospholipids, trehalose lipids, trehalose dimycolates, trehalose monomycolates, mannosylerythritol lipids (MEL), cellobiose lipids, ustilagic acids and/or sophorolipids (SLP).

In one embodiment, polyketides comprise compounds containing carbonyl and methylene groups (β-polyketones), preferably, compounds comprising alternating carbonyl and methylene groups. Polyketides include, but are not limited to erythromycin, clarithromycin, azithromycin, nystatin, avermectin, spiramycin, tetracyclines, tacrolimus, rapamycin, radicicol, pochonin, lovastatin, doxorubicin, emodin, albendazole, mebendazole, diethylcarbamazine, ivermectin, praziquantel and spinosyn A.

In one embodiment, the subject invention provides a method for isolating/extracting one or more microbial metabolites with a high purity, the method comprising:

1) cultivating a metabolite-producing microorganism to produce a microorganism culture broth, said microorganism culture broth comprising liquid fermentation medium, microorganism cells and one or more microbial metabolites;

2) mixing the culture broth with one or more solvents, preferably, non-polar solvents such as, e.g., ethyl acetate;

3) centrifuging the mixture of step 2) to cause phase separation in the mixture, wherein a non-polar layer, an interphase layer, a water layer and a cell layer are separated;

4) separately collecting one or more layers selected from the non-polar layer, interphase layer, water layer and cell layer;

5) independently centrifuging the collected one or more layers of step 4) and independently collecting each of the supernatants or the solvent layers from the one or more collected layers; and

6) obtaining one or more microbial metabolites from each of the non-polar layer, interphase layer, water layer and cell layer, for example, by evaporation.

In one embodiment, the water layer is collected in step 4). In one embodiment, the method according to the subject invention provides steps for purifying, isolating or extracting a single-molecule product of the microbial metabolite from the water layer obtained in step 4). Preferably, the microbial metabolite obtained from the water layer is a hydrophilic molecule or water-soluble molecule. The method for obtaining the microbial metabolite comprises evaporating the water content in the collected supernatant from the above-mentioned step 5), for example through a rotary evaporator, and obtaining the single-molecule product of the microbial metabolite at a high level of purity.

In one embodiment, the method according to the subject invention also provides steps for isolating, extracting, obtaining and/or purifying one or more single-molecule products of microbial metabolites from the water layer. The method comprises a plurality of isolating/extracting/purifying cycles using solvents with increased non-polarity for obtaining each of the one or more microbial metabolites in the water layer collected in the above-mentioned step 4) and/or 5). In one embodiment, each isolating/extracting/purifying cycle comprises:

i) obtaining an evaporated product of the water layer;

ii) mixing the evaporated product of the water layer with a solvent, for example, an alcohol such as methanol;

iii) centrifuging the mixture from step ii) and collecting the supernatant or the solvent layer; and

iv) obtaining the single-molecule product of a microbial metabolite by evaporating the solvent, for example, using a rotary evaporator;

wherein each cycle uses a different solvent that is more non-polar than the solvent used in the previous cycle. Preferably, the solvents may be used in an order of: methanol, ethanol, 1-propanol, 1-butanol, 1-hexanol, 1-heptanol, 2-propanol, 2-butanol, acetone, and chloroform.

In one embodiment, the non-polar layer is collected in the above-mentioned step 4). In one embodiment, the method according to the subject invention provides steps for isolating or extracting a single-molecule product of the microbial metabolite from the non-polar layer obtained in the above-mentioned step 4). The method for obtaining the microbial metabolite comprises evaporating the non-polar solvent in the collected supernatant or the solvent layer from the above-mentioned step 5), for example through a rotary evaporator, and obtaining the single-molecule product of the microbial metabolite at a high level of purity.

In one embodiment, the method according to the subject invention also provides steps for isolating, extracting, obtaining and/or purifying one or more single-molecule products of microbial metabolites from the non-polar layer obtained in the above-mentioned step 5). The method comprises a plurality of isolating/extracting/purifying cycles using solvents with increased polarity for obtaining each of the one or more microbial metabolites in the non-polar layer collected in the above-mentioned step 4) and/or 5). In one embodiment, each isolating/extracting/purifying cycle comprises:

i) obtaining an evaporated product of the non-polar layer;

ii) mixing the evaporated product of the non-polar layer with a solvent, for example, a non-polar solvent, such as chloroform;

iii) centrifuging the mixture from step ii) and collecting the supernatant or the solvent layer; and

iv) obtaining the single-molecule product of a microbial metabolite by evaporating the solvent, for example, using a rotary evaporator;

wherein each cycle uses a different solvent that is more polar than the solvent used in the previous cycle. Preferably, the solvents may be used in an order of: chloroform, acetone, 2-butanol, 2-propanol, 1-heptanol, 1-hexanol, 1-butanol, 1-propanol, ethanol, and methanol.

In one embodiment, the interphase layer is collected in the above-mentioned step 4). In one embodiment, the method according to the subject invention further provides steps for extracting one or more single-molecule products of microbial metabolites from the interphase layer obtained in the above-mentioned step 4). The method comprises a plurality of extracting/purifying cycles using solvents with increased polarity for obtaining each of the one or more microbial metabolites in the interphase layer. In one embodiment, each extracting/purifying cycle comprises:

i) obtaining an evaporated product of the interphase layer;

ii) mixing the evaporated product of the interphase layer with a solvent, for example, a non-polar solvent, such as chloroform;

iii) centrifuging the mixture from step ii) and collecting the supernatant or the solvent layer; and

iii) obtaining the single-molecule product of a microbial metabolite by evaporating the solvent, for example, using a rotary evaporator;

wherein each cycle uses a different solvent that is more polar than the solvent used in the previous cycle. Preferably, the solvents may be used in an order of: chloroform, acetone, 2-butanol, 2-propanol, 1-heptanol, 1-hexanol, 1-butanol, 1-propanol, ethanol, and methanol.

In one embodiment, the cell layer is collected in the above-mentioned step 4). In one embodiment, the method according to the subject invention further provides steps for isolating/extracting one or more single-molecule products of microbial metabolites from the cell layer obtained in the above-mentioned step 4). The method comprises a plurality of isolating/extracting/purifying cycles using solvents with increased non-polarity for obtaining each of the one or more microbial metabolites in the cell layer. In one embodiment, each isolating/extracting/purifying cycle comprises:

i) obtaining an evaporated product of the cell layer;

ii) mixing the evaporated product of the cell layer with a solvent, for example, an alcohol such as methanol;

iii) centrifuging the mixture from step ii) and collecting the supernatant or the solvent layer; and

iii) obtaining the single-molecule product of a microbial metabolite by evaporating the solvent, for example, using a rotary evaporator;

wherein each cycle uses a different solvent that is more non-polar than the solvent used in the previous cycle. Preferably, the solvents may be used in an order of: methanol, ethanol, 1-propanol, 1-butanol, 1-hexanol, 1-heptanol, 2-propanol, 2-butanol, acetone, and chloroform.

Advantageously, the methods according to the subject invention can facilitate purification of microbial metabolites such as SLP derivatives to very high purity, for example, 95%, 98% or greater. Additionally, the methods and equipment of the subject invention reduce the capital and labor costs, as well as the environmental impacts and health hazards, of producing microorganisms and purifying their metabolites on a large scale.

### DETAILED DESCRIPTION OF THE INVENTION

The subject invention provides materials and methods for producing, extracting and purifying single-molecule products. Specifically, the subject invention provides materials and methods for extracting microbial metabolites at a high level of purity, a purity of, for example, at least 70% by weight, at least 75% by weight, at least 80% by weight, at least 85% by weight, at least 90% by weight, at least 95% by weight, at least 98% by weight, at least 99% by weight, at least 99.9% by weight, or at least 99.99% by weight. Advantageously, the subject invention is suitable for industrial scale production of purified microbial metabolites, and uses safe and environmentally-friendly materials and processes.

The subject invention also provides materials and methods for producing, extracting and purifying a single-molecule product that is substantially free of other compounds or cellular material. The purified or isolated single-molecule product may have a purity of, for example, at least 80% by weight, at least 85% by weight, at least 90% by weight, at least 95% by weight, at least 98% by weight, or at least 99% by weight, at least 99.9% by weight or at least 100% by weight.

In one embodiment, the single-molecule product contains a single microbial metabolite produced by microorganisms. In one embodiment, the microbial metabolite is selected from primary metabolites such as saccharides, amino acids, and lipids and secondary metabolites such as biosurfactants, polyketides, alkaloids, flavonoids, glycosides, quinolones, terpenoids, peptides and growth factors. In a further embodiment, the microbial metabolite is selected from biosurfactants and polyketides.

In one embodiment, the biosurfactants include glycolipids, lipopeptides, flavolipids, phospholipids, and fatty acid esters, and high molecular weight polymers such as lipoproteins, lipopolysaccharide-protein complexes, and polysaccharide-protein-fatty acid complexes. Preferably, the biosurfactants are glycolipids that comprise a carbohydrate and at least one fatty acid. Glycolipids include, for example, rhamnolipids (RLP), rhamnose-d-phospholipids, trehalose lipids, trehalose dimycolates, trehalose monomycolates, mannosylerythritol lipids (MEL), cellobiose lipids, ustilagic acids and/or sophorolipids (SLP). More preferably, the biosurfactant is SLP.

Sophorolipids are glycolipid biosurfactants produced by, for example, various yeasts of the Starmerella clade. SLP consist of a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid is generally 16 or 18 carbon atoms, and may contain one or more unsaturated bonds. Furthermore, the sophorose residue can be acetylated on the 6- and/or 6′-position(s). The fatty acid carboxyl group can be free (acidic or linear form) or internally esterified at the 4″-position (lactonic form). S. bombicola produces a specific enzyme, called S. bombicola lactone esterase, which catalyzes the esterification of linear SLP to produce lactonic SLP.

Due to the structure and composition of SLP, these biosurfactants have excellent surface and interfacial tension reduction properties, as well as other beneficial biochemical properties, which can be useful as a replacement for chemical surfactants in applications such as large scale industrial and agriculture uses, cosmetics, household products, health, medical and pharmaceutical fields, and oil and gas recovery.

In some embodiments, the invention is useful for production of high purity fatty acid esters, such as, for example, fatty acid methyl esters, fatty acid ethyl esters, fatty acid isopropyl esters, fatty acid butyl esters, and/or fatty acid hexyl esters.

In one embodiment, polyketides are compounds containing carbonyl and methylene groups (β-polyketones), preferably, compounds comprising alternating carbonyl and methylene groups. Polyketides include, but are not limited to, erythromycin, clarithromycin, azithromycin, nystatin, avermectin, spiramycin, tetracyclines, tacrolimus, rapamycin, radicicol, pochonin, lovastatin, doxorubicin, emodin, azitromycin, clarithromycin, roxithromycin, albendazole, mebendazole, diethylcarbamazine, ivermectin, praziquantel and spinosyn A.

In a preferred embodiment, the polyketide is erythromycin having a structure of:

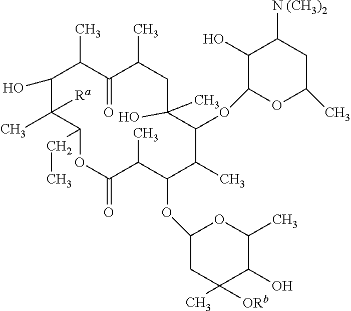

wherein R^{a} is OH or H and R^{b} is CH₃ or H. Specifically, the polyketide is selected from erythromycin A, B, C, or D.

### Selected Definitions

As used herein, the term “sophorolipid,” “sophorolipid molecule,” “SLP” or “SLP molecule” includes all forms, and isomers thereof, of SLP molecules, including, for example, acidic (linear) SLP (ASL) and lactonic SLP (LSL). Further included are mono-acetylated SLP, di-acetylated SLP, esterified SLP, SLP with varying hydrophobic chain lengths, SLP with fatty acid-amino acid complexes attached, and others as are described within in this disclosure.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The microbes may be present in or removed from the composition. The microbes can be present, with broth in which they were grown, in the microbe-based composition. The cells may be present at, for example, a concentration of at least 1×10⁴, 1×10⁵, 1×10⁶, 1×10⁷, 1×10⁸, 1×10⁹, 1×10¹⁰, 1×10¹¹, 1×10¹², or more CFU per milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, such as plant hormones, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “harvested” refers to removing some or all of a microbe-based composition from a growth vessel.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 98%, by weight the compound of interest. For example, a purified compound is one that is at least 80%, 85%, 90%, 91%, 92%, 93%, 94%, 95%, 96%, 97%, 98%, 99%, 99.9%, 99.99% or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, biopolymers, polyketides and biosurfactants.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the alteration is plus or minus 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, “surfactant” means a compound that lowers the surface tension (or interfacial tension) between two liquids or between a liquid and a solid. Surfactants act as, e.g., detergents, wetting agents, emulsifiers, foaming agents, and/or dispersants. A “biosurfactant” is a surface-active substance produced by a living cell.

As used herein, a “broth,” “culture broth,” or “fermentation broth” refers to a culture medium comprising at least nutrients and microorganism cells.

Unless the context requires otherwise, the phrases “fermenting,” “fermentation process” or “fermentation reaction” and the like, as used herein, are intended to encompass both the growth phase and product biosynthesis phase of the process.

As used herein, the term “reactor,” “bioreactor,” or “fermentation reactor” includes a fermentation device consisting of one or more vessels and/or towers or piping arrangements. Examples of such reactor includes, but are not limited to, the Continuous Stirred Tank Reactor (CSTR), Immobilized Cell Reactor (ICR), Trickle Bed Reactor (TBR), Bubble Colunm, Gas Lift Fermenter, Static Mixer, or other vessel or other device suitable for gas-liquid contact. In some embodiments, the bioreactor may comprise a first growth reactor and a second fermentation reactor. As such, when referring to the addition of substrate to the bioreactor or fermentation reaction, it should be understood to include addition to either or both of these reactors where appropriate.

As used herein, a “recombinant microorganism” is a microorganism that has undergone intentional genetic modification when compared to a parental microorganism. A “genetic modification” should be taken broadly and includes, for example, insertion, deletion or substitution of nucleic acids.

A “parental microorganism” is a microorganism used to generate a recombinant microorganism of the invention. The parental microorganism may be one that occurs in nature (i.e., a wild type microorganism) or one that has been previously modified (i.e., it is a recombinant microorganism). The recombinant microorganisms of the invention may be modified to express or over-express one or more proteins/enzymes that were not expressed or over-expressed to a desired level in the parental microorganism, or may be modified to exhibit increased productivity of metabolites.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Microorganisms

The microorganisms according to the subject invention can be, for example, bacteria, yeasts, fungi or multicellular organisms. The microorganisms utilized according to the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a parental microorganism, wherein the mutant has one or more genetic variations (e.g., a point mutation, missense mutation, nonsense mutation, deletion, duplication, frameshift mutation or repeat expansion) as compared to the parental microorganism. Procedures for making mutants are well known in the microbiological art. For example, UV mutagenesis and nitrosoguanidine are used extensively toward this end.

In one embodiment, the microorganisms are bacteria, including gram-positive and gram-negative bacteria. These bacteria may be, but are not limited to, for example, Saccharopolyspora erythraea, Escherichia coli, Rhizobium (e.g., Rhizobium japonicum, Sinorhizobium meliloti, Sinorhizobium fredii, Rhizobium leguminosarum biovar trifolii, and Rhizobium etli), Bradyrhizobium (e.g., Bradyrhizobium japonicum, and B. parasponia), Bacillus (e.g., Bacillus subtilis, Bacillus firmus, Bacillus laterosporus, Bacillus megaterium, Bacillus amyloliquifaciens), Azobacter (e.g., Azobacter vinelandii, and Azobacter chroococcum), Arhrobacter (e.g. Agrobacterium radiobacter), Pseudomonas (e.g., Pseudomonas chlororaphis subsp. aureofaciens (Kluyver)), Azospirillium (e.g., Azospirillumbrasiliensis), Azomonas, Derxia, Beijerinckia, Nocardia, Klebsiella, Clavibacter (e.g., C. xyli subsp. xyli and C. xyli subsp. cynodontis), cyanobacteria, Pantoea (e.g., Pantoea agglomerans), Sphingomonas (e.g., Sphingomonas paucimobilis), Streptomyces (e.g., Streptomyces griseochromogenes, Streptomyces qriseus, Streptomyces cacaoi, Streptomyces aureus, and Streptomyces kasugaenis), Streptoverticillium (e.g., Streptoverticillium rimofaciens), Ralslonia (e.g., Ralslonia eulropha), Rhodospirillum (e.g., Rhodospirillum rubrum), Xanthomonas (e.g., Xanthomonas campestris), Erwinia (e.g., Erwinia carotovora), Clostridium (e.g., Clostridium bravidaciens, and Clostridium malacusomae) and combinations thereof.

In preferred embodiments, the microorganism is any yeast or fungus. Examples of yeast and fungus species suitable for use according to the current invention, include, but are not limited to, Acaulospora, Aspergillus, Aureobasidium (e.g., A. pullulans), Blakeslea, Candida (e.g., C. albicans, C. apicola), Cryptococcus, Debaryomyces (e.g., D. hansenii), Entomophthora, Fusarium, Hanseniaspora (e.g., H. uvarum), Hansenula, Issatchenkia, Kluyveromyces, Mortierella, Mucor (e.g., M. piriformis), Meyerozyma (e.g., M. guilliermondii), Penicillium, Phythium, Phycomyces, Pichia (e.g., P. anomala, P. guilliermondii, P. occidentalis, P. kudriavzevii), Pseudozyma (e.g., P. aphidis), Rhizopus, Saccharomyces (S. cerevisiae, S. boulardii sequela, S. torula), Starmerella (e.g., S. bombicola), Torulopsis, Thraustochytrium, Trichoderma (e.g., T. reesei, T. harzianum, T. vixens), Ustilago (e.g., U. maydis), Wickerhamomyces (e.g., W. anomalus), Williopsis, and Zygosaccharomyces (e.g., Z. bailii).

In preferred embodiments, microorganism is a Starmerella spp. yeast and/or Candida spp. yeast, e.g., Starmerella (Candida) bombicola, Candida apicola, Candida batistae, Candida floricola, Candida riodocensis, Candida stellate and/or Candida kuoi. In a specific embodiment, the microorganism is Starmerella bombicola, e.g., strain ATCC 22214.

In preferred embodiments, the sophorolipid-producing yeast is Starmerella bombicola, or another member of the Starmerella and/or Candida clades. For example, S. bombicola strain ATCC 22214 can be used according to the subject methods.

### Methods

The subject invention provides materials and methods for producing, isolating, extracting and purifying microbial metabolites at a high level of purity. Specifically, the subject invention provides materials and methods for the purification of microbial metabolites to a purity of, for example, at least 80% by weight, and preferably at least 95% by weight or more.

The subject invention also provides materials and methods for producing, isolating, extracting, obtaining and purifying a single-molecule product from a biosynthetic mixture. The single-molecule product is substantially free of other compounds or cellular material. The purified or isolated single-molecule product may have a purity of, for example, at least 80% by weight, at least 85% by weight, at least 90% by weight, at least 95% by weight, at least 98% by weight, or at least 99% by weight, at least 99.9% by weight or at least 100% by weight.

In one embodiment, the method for extracting a microbial metabolite with a high purity comprises cultivating a metabolite-producing microorganism to produce a microorganism culture broth, said culture broth comprising liquid fermentation medium, cells and one or more microbial metabolites; and extracting a single microbial metabolite from the culture. In certain embodiments, more than one microbial metabolite is produced by the microorganism; thus, each of the more than one microbial metabolite can also be extracted from the culture with high purities.

The subject invention provides for selective molecule separation. In certain embodiments, the metabolite(s) of interest are not strictly hydrophilic or hydrophobic, but instead are characterized as amphiphilic with a tendency to be more hydrophilic or hydrophobic. Thus, a mixture of molecules can be treated with solutions having components with slightly different hydrophobicity/hydrophilicity, so that the molecule of interest will gravitate to the component having the most preferable character relative to the other components. When repeated over the course of multiple cycles, the process leads to a single molecule present in its suitable solvent.

In one embodiment, the method comprises filling a fermentation reactor with a liquid nutrient medium; inoculating the reactor with a microorganism that produces one or more microbial metabolites of interest, for example, a sophorolipid-producing yeast, to produce a microorganism culture; and cultivating the microorganism culture under conditions favorable for production of one or more microbial metabolites, e.g., SLP, erythromycin and/or a fatty acid isopropyl ester.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, metabolite concentration, and/or visible oil level monitoring. For example, in one embodiment, sampling can occur every 24 hours.

The microbial inoculant according to the subject methods preferably comprises cells and/or propagules of the desired microorganism, which can be prepared using any known fermentation method. The inoculant can be pre-mixed with water and/or a liquid growth medium, if desired.

In certain embodiments, the cultivation method utilizes submerged fermentation in a liquid growth medium. In one embodiment, the liquid growth medium comprises a carbon source. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, soybean oil, rice bran oil, olive oil, corn oil, sunflower oil, sesame oil, and/or linseed oil; powdered molasses, etc. These carbon sources may be used independently or in a combination of two or more. In preferred embodiments, a hydrophilic carbon source, e.g., glucose, and a hydrophobic carbon source, e.g., oil or fatty acids, are used.

In one embodiment, the liquid growth medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid. In certain embodiments, dissolved oxygen (DO) levels are maintained at about 25% to about 75%, about 30% to about 70%, about 35% to about 65%, about 40% to about 60%, or about 50% of air saturation.

In some embodiments, the method for cultivation may further comprise adding additional acids and/or antimicrobials in the liquid medium before and/or during the cultivation process. Antimicrobial agents or antibiotics (e.g., streptomycin, oxytetracycline) are used for protecting the culture against contamination. In some embodiments, however, the metabolites produced by the yeast culture provide sufficient antimicrobial effects to prevent contamination of the culture.

In one embodiment, prior to inoculation, the components of the liquid culture medium can optionally be sterilized. In one embodiment, sterilization of the liquid growth medium can be achieved by placing the components of the liquid culture medium in water at a temperature of about 85-100° C. In one embodiment, sterilization can be achieved by dissolving the components in 1 to 3% hydrogen peroxide in a ratio of 1:3 (w/v).

In one embodiment, the equipment used for cultivation is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Gaskets, openings, tubing and other equipment parts can be sprayed with, for example, isopropyl alcohol. Air can be sterilized by methods know in the art. For example, the ambient air can pass through at least one filter before being introduced into the vessel. In other embodiments, the medium may be pasteurized or, optionally, no heat at all added, where the use of pH and/or low water activity may be exploited to control unwanted microbial growth.

The pH of the culture should be suitable for the microorganism of interest. In some embodiments, the pH is about 2.0 to about 10.0, about 2.0 to about 9.5, about 2.0 to about 9.0, about 2.0 to about 8.5, about 2.0 to about 8.0, about 2.0 to about 7.5, about 2.0 to about 7.0, about 3.0 to about 7.5, about 4.0 to about 7.5, about 5.0 to about 7.5, about 5.5 to about 7.0, about 6.5 to about 7.5, about 3.0 to about 5.5, about 3.25 to about 4.0, or about 3.5. Buffers, and pH regulators, such as carbonates and phosphates, may be used to stabilize pH near a preferred value. In certain embodiments, a base solution is used to adjust the pH of the culture to a favorable level, for example, a 15% to 30%, or a 20% to 25% NaOH solution. The base solution can be included in the growth medium and/or it can be fed into the fermentation reactor during cultivation to adjust the pH as needed.

In one embodiment, the method of cultivation is carried out at about 5° to about 100° C., about 15° to about 60° C., about 20° to about 50° C., about 20° to about 45° C., about 25° to about 40° C., about 25° to about 37° C., about 25° to about 35° C., about 30° to about 35° C., about 24° to about 28° C., or about 22° to about 25° C. In one embodiment, the cultivation may be carried out continuously at a constant temperature. In another embodiment, the cultivation may be subject to changing temperatures.

According to the subject invention, the microorganisms can be incubated in the fermentation system for a time period sufficient to achieve a desired effect, e.g., production of a desired amount of cell biomass or a desired amount of one or more microbial growth by-products such as microbial metabolites. The microbial growth by-product(s) produced by microorganisms may be retained in the microorganisms and/or secreted into the growth medium. The biomass content may be, for example from 5 g/l to 180 g/l or more, or from 10 g/I to 150 WI.

In certain embodiments, fermentation of the yeast culture occurs for about 100 to 150 hours, or about 115 to about 125 hours, or about 120 hours. In some embodiments, the fermentation cycle is ended once the glucose and/or oil concentrations in the medium are exhausted. In some embodiments, the end of the fermentation cycle is determined to be a time point when the microorganisms have begun to consume trace amounts of SLP.

In one embodiment, the subject invention provides methods for isolating/extracting/purifying one or more microbial metabolites with a high purity, the method comprising:

1) cultivating a metabolite-producing microorganism to produce a microorganism culture broth;

2) mixing the culture broth with one or more solvents, preferably, non-polar solvents; 3) centrifuging the mixture of step 2) to cause phase separation in the mixture, wherein a solvent layer (e.g., non-polar layer), an interphase layer, a water layer and a cell layer are separated;

4) separately collecting one or more layers selected from the solvent layer (e.g., non-polar layer), interphase layer, water layer and cell layer;

5) independently centrifuging each of the collected layers and independently collecting each of the supernatants or the solvent layers from the one or more collected layers; and

6) obtaining one or more microbial metabolites from each of the non-polar layer, interphase layer, water layer and cell layer, for example, by evaporation.

In one embodiment, the method comprises mixing the culture broth with one or more non-polar solvents. Examples of non-polar chemicals as solvent for the subject invention include, but are not limited to, ethyl acetate, polyethylene glycol, tetrahydrofuran, dichloromethane, chloroform, diethyl ether, pentane, hexane, benzene, toluene, cyclohexane, heptane, carbon disulfide, p-xylene, benzene, ether, methyl t-butyl ether (MTBE), diethylamine, dioxane, N,N-dimethylaniline, chlorobenzene, anisole, tetrahydrofuran (THF), ethyl benzoate, dimethoxyethane, diglyme, methyl acetate, carbon tetrachloride, 3-pentanone, 1,1-dichloroethane, di-n-butyl phthalate, cyclohexanone, and pyridine. Preferably, the non-polar solvent is ethyl acetate.

In one embodiment, the culture broth is mixed with the solvent, preferably, non-polar solvent, at a ratio from 1:10 (v/v) to 10:1 (v/v), from 1:5 (v/v) to 5:1 (v/v), and from 1:2 (v/v) to 2:1 (v/v). Specifically, the culture broth is mixed with the non-polar solvent at a ratio of, for example, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 2:9, 2:7, 2:5, 2:3, 3:8, 3:7, 3:5, 3:4, 3:2, 4:9, 4:7, 4:5, 5:9, 5:8, 5:7, 5:6, 5:4, 5:3, 5:2, 6:7, 6:5, 7:9, 7:8, 7:6, 7:5, 7:4, 7:3, 7:2, 8:9, 8:7, 8:5, 8:3, 9:8, 9:7, 9:5, 9:4, or 9:2. Preferably, the culture broth is mixed with the non-polar solvent at a ratio of 1:1.

In one embodiment, the culture broth is mixed with the solvent, preferably, non-polar solvent for at least 0.5, 1, 2, 5, 10, 15, 20, 24, 30, 36, 42, 48, 54, or 60 hours. Preferably, the culture broth is mixed with the solvent, preferably, non-polar solvent for at least 24 hours.

The mixture of culture broth and solvent is then centrifuged at a speed for a time period that is enough to cause phase separation in the mixture. Preferably, the mixture is centrifuged at 10000 g for 30 min. As a result, a non-polar solvent layer, an interphase layer, a water phase layer and a cell layer form. The non-polar layer, interphase layer, water phase layer and cell layers are collected separately.

In one embodiment, the non-polar layer and water layer are each collected separately and centrifuged at a speed for a time period that is sufficient for precipitation. Such speed and/or time period for each of non-polar layer and water layer may be the same or different. In a preferred embodiment, the non-polar and water layers are each centrifuged at 10000 g for 30 min. The supernatant or the solvent layer from each of the non-polar and water layers is collected. The precipitate from the non-polar layer is combined with the previously collected interphase layer while the precipitate from the water layer is combined with the previously collected cell layer.

In one embodiment, the method comprises obtaining a single-molecule product of the microbial metabolite by evaporating the collected supernatant of the water layer, for example, using a rotary evaporator.

According to the subject invention, the temperature for the rotary evaporator has a range of, for example, from about 4° C. to about 60° C., from about 10° C. to about 60° C., from about 15° C. to about 55° C., from about 20° C. to about 50° C., from about 20° C. to about 45° C., from about 25° C. to about 45° C., from about 30° C. to about 45° C., or from about 35° C. to about 45° C. Preferably, the temperature condition for rotary evaporator is no higher than 45° C. to prevent degradation of the product.

According to the subject invention, the pressure for the rotary evaporator has a range of, for example, from about 1 mbar to 1 bar, from about 2 mbar to 900 mbar, from about 2 mbar to 800 mbar, from about 2 mbar to 700 mbar, from about 2 mbar to 600 mbar, from about 5 mbar to 500 mbar, from about 5 mbar to 400 mbar, from about 5 mbar to 300 mbar, from about 5 mbar to 200 mbar, from about 5 mbar to 100 mbar, from about 5 mbar to 100 mbar, from about 5 mbar to 90 mbar, from about 5 mbar to 80 mbar, from about 5 mbar to 70 mbar, from about 5 mbar to 60 mbar, or from about 5 mbar to 50 mbar.

According to the subject invention, the relative centrifugal force (rcf) or the degree of acceleration used in centrifugation has a range of, for example, from about 1 000 g to about 1000000 g, from about 1000 g to about 500000 g, from about 1000 g to about 200000 g, from about 1000 g to about 100000 g, from about 1000 g to about 50000 g, from about 1000 g to about 20000 g, from about 1000 g to about 10000 g, from about 2000 g to about 20000 g, from about 5000 g to about 20000 g, from about 10000 g to about 20000 g, or from about 10000 g to about 15000 g.

According to the subject invention, the time period for centrifugation has as range of, for example, from about 1 min to about 360 min, from about 1 min to about 300 min, from about 5 min to about 240 min, from about 10 min to about 210 min, from about 10 min to about 180 min, from about 15 min to about 150 min, from about 15 min to about 120 min, from about 15 min to about 90 min, from about 15 min to about 60 min, from about 15 min to about 45 min, or from about 20 min to about 30 min. Preferably, each centrifugation runs for about 15 min to about 45 min. More preferably, each centrifugation runs for 30 min.

In one embodiment, the water layer is collected in the above-mentioned step 4). In one embodiment, the subject invention provides methods for isolating/extracting/purifying a single-molecule product of the microbial metabolite from the water layer obtained in step 4). Preferably, the microbial metabolite obtained from the water layer is a hydrophilic molecule or water-soluble molecule. The method for obtaining the microbial metabolite comprises evaporating the water content in the collected supernatant from the above-mentioned step 5), for example, through a rotary evaporator, and obtaining the single-molecule product of the microbial metabolite at a high level of purity.

In one embodiment, the method according to the subject invention also provides steps for isolating, extracting, obtaining and/or purifying one or more, two or more, three or more, or four or more single-molecule products of microbial metabolites from the water layer. The method comprises a plurality of isolating/extracting/purifying cycles using solvents with increased non-polarity for obtaining each of the microbial metabolites in the water layer collected in the above mentioned step 4) and/or 5). In one embodiment, each isolating/extracting/purifying cycle comprises:

i) obtaining an evaporated product of the water layer;

ii) mixing the evaporated product of the water layer with a solvent, for example, an alcohol such as methanol;

iii) centrifuging the mixture from step ii) and collecting the supernatant or the solvent layer; and

iv) obtaining the single-molecule product of a microbial metabolite by evaporating the solvent, for example, using a rotary evaporator;

wherein each cycle uses a different solvent that is more non-polar than the solvent used in the previous cycle. Preferably, the solvents may be used in an order of: methanol, ethanol, 1-propanol, 1-butanol, 1-hexanol, 1-heptanol, 2-propanol, 2-butanol, acetone, and chloroform.

In one embodiment, the method of the subject invention comprises further steps for purifying the obtained product of microbial metabolite to eliminate other compounds, for example, glucose such as caramelized glucose. The method may further comprise mixing the evaporated product from the water layer, as noted above, with 50%, 60%, 70%, 80%, 90%, 95%, 99%, or 100% alcohol, centrifuging the mixture of the evaporated product from the water layer and alcohol, collecting the supernatant, and obtaining the single-molecule product by evaporating the alcohol in the collected supernatant using a rotary evaporator.

In one embodiment, the evaporated product from water layer is mixed with alcohol at a ratio from 1:10 (v/v) to 10:1 (v/v), from 1:5 (v/v) to 5:1 (v/v), and from 1:2 (v/v) to 2:1 (v/v). Specifically, the evaporated product from water layer is mixed with alcohol at a ratio of, for example, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 2:5, 2:3, 3:5, 3:4, or 3:2. Preferably, the evaporated product from water layer is mixed with alcohol at a ratio of 1:1.

In one embodiment, the evaporated product from the water layer is mixed with alcohol at room temperatures or, for higher yields, at lower temperatures. Exemplary temperature is about 4° to about 30° C., about 5° to about 25° C., about 10° to about 25° C., about 15° to about 25° C., about 20° to about 25° C., or about 22° to about 25° C. In one embodiment, the mixing may be carried out continuously at a constant temperature. In another embodiment, the mixing may be subject to changing temperatures.

In one embodiment, the evaporated product from the water layer is mixed with alcohol, for at least 0.5, 1, 5, 10, 15, 20, 24, 30, 36, 42, 48, 54, or 60 hours. Preferably, the evaporated product from the water layer is mixed with alcohol for 24 hours.

Advantageously, mixing the evaporated product from the water layer with alcohol can remove compounds that do not dissolve in alcohol while the single-molecule product of microbial metabolite of the subject invention can dissolve in alcohol.

In one embodiment, the mixture of the evaporated product from the water layer and alcohol is centrifuged at a speed, and for a time period, that is sufficient for precipitating the compounds that do not dissolve in alcohol. Preferably, the mixture is centrifuged at 10000 g for 30 min. The supernatant, i.e., the alcohol layer, and the precipitates are collected separately. The single-molecule product can be then obtained by evaporating the alcohol in the collected supernatant using a rotary evaporator.

In one embodiment, “alcohol,” according to the subject invention, includes, but is not limited to, methanol, ethanol, propanol, butanol, pentanol, hexanol, heptanol, octanol, hexadecanol, ethylene glycol, propylene glycol, glycerol, erythritol, xylitol, and mannitol.

In some embodiments, two or more microbial metabolites are present in the evaporated product from the alcohol layer. Such evaporated product can be mixed with another solvent, e.g., an alcohol, which is more non-polar than the alcohol used to mix with the evaporated product from the water layer. Only the more non-polar compound will be dissolved and thus, centrifugation, for example, at 10000 g for 30 min., will isolate the less soluble compounds through precipitation. Further evaporation of the more non-polar layer will arrive at a product containing only a single molecule.

In a specific embodiment, methanol is mixed with the evaporated product from the water layer to obtain the evaporated product from the methanol layer. Such product of the methanol layer can then be mixed with, for example, 100% ethanol, which is more non-polar than methanol and will dissolve the compound that is more non-polar among two or more microbial metabolites. For example, when both monoacetylated and diacetylated linear SLP are present in the evaporated product from the methanol layer, such product can be mixed with 100% ethanol, which first dissolves the more non-polar product, i.e., diacetylated linear SLP. Thus, only diacetylated linear SLP will dissolve in ethanol. Centrifuging such product will lead to the precipitation of monoacetylated linear SLP, while diacetylated linear SLP will be obtained from the supernatant of the ethanol layer. Further evaporation of such ethanol layer will arrive at a product containing only diacetylated linear SLP.

In some embodiments, two or more microbial metabolites are present in the evaporated product from the water layer. The above-mentioned step can be repeated by mixing the product with different solvents by increasing their non-polarity. The solvents may be used in an order of, for example, methanol, ethanol, 1-propanol, 1-butanol, 1-hexanol, 1-heptanol, 2-propanol, 2-butanol, acetone, and chloroform.

In one embodiment, the non-polar layer is collected in the above-mentioned step 4). In one embodiment, the method according to the subject invention provides steps for isolating/extracting a single-molecule product of the microbial metabolite from the non-polar layer obtained in the above-mentioned step 4). The method for obtaining the microbial metabolite comprises evaporating the non-polar solvent in the collected supernatant or the solvent layer from the above-mentioned step 5), for example, through a rotary evaporator, and obtaining the single-molecule product of the microbial metabolite at a high level of purity.

In one embodiment, the method according to the subject invention also provides steps for isolating, extracting, obtaining and/or purifying one or more single-molecule products of microbial metabolites from the non-polar layer obtained in the above-mentioned step 5). The method comprises a plurality of isolating/extracting/purifying cycles using solvents with increased polarity for obtaining each of the one or more microbial metabolites in the non-polar layer collected in the above-mentioned step 4) and/or 5). In one embodiment, each isolating/extracting/purifying cycle comprises:

i) obtaining an evaporated product of the non-polar layer;

ii) mixing the evaporated product of the non-polar layer with a solvent, for example, a non-polar solvent, such as chloroform;

iii) centrifuging the mixture from step ii) and collecting the supernatant or the solvent layer; and

iv) obtaining the single-molecule product of a microbial metabolite by evaporating the solvent, for example, using a rotary evaporator;

wherein each cycle uses a different solvent that is more polar than the solvent used in the previous cycle. Preferably, the solvents may be used in an order of: chloroform, acetone, 2-butanol, 2-propanol, 1-heptanol, 1-hexanol, 1-butanol, 1-propanol, ethanol, and methanol.

In one embodiment, the method for obtaining a single-molecule product from the non-polar layer comprises obtaining an evaporated product from the non-polar layer by evaporating the collected supernatant of the non-polar layer, for example, using a rotary evaporator, mixing the evaporated product of the non-polar layer with a second non-polar solvent, such as, for example, chloroform, centrifuging the mixture, for example, at 10000 g for 30 min, to produce a top and bottom layer, and collecting the top and bottom layers separately, and evaporating the top layer to obtain a single-molecule product. The bottom layer can also be evaporated to analyze the contents thereof.

In one embodiment, the evaporated product from the non-polar layer is mixed with a second non-polar solvent such as chloroform at a ratio from 1:10 (v/v) to 10:1 (v/v), from 1:5 (v/v) to 5:1 (v/v), and from 1:2 (v/v) to 2:1 (v/v). Specifically, the evaporated product from the non-polar layer is mixed with the second non-polar solvent, e.g., chloroform, at a ratio of, for example, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 2:5, 2:3, 3:5, 3:4, or 3:2. Preferably, the evaporated product from the non-polar layer is mixed with the second non-polar solvent at a ratio of 1:1.

In one embodiment, the evaporated product from the non-polar layer is mixed with the second non-polar solvent, for at least 1, 5, 10, 15, 20, 24, 30, 36, 42, 48, 54, or 60 hours. Preferably, the evaporated product from the water layer is mixed with alcohol for 24 hours.

In some embodiments, two or more microbial metabolites are present in the evaporated product from the non-polar layer. The evaporated product of the top layer can then be mixed with another solvent that is more polar than the second non-polar solvent so that only the more polar compound will be dissolved. For example, if chloroform is used as the second non-polar solvent, the evaporated product of the top layer can then be mixed with, for example, acetone, a more polar solvent than chloroform. In some embodiments, the evaporated product of the bottom layer can also be treated in this manner. After centrifugation at, for example 10000 g for 30 min, the supernatant is collected and evaporated to obtain a single-molecule product and the less soluble compounds are isolated through precipitation.

In certain embodiments, the above-mentioned step can be repeated by mixing the evaporated product with different solvents by increasing their polarity. The solvents may be used in an order of, for example, chloroform, acetone, 2-butanol, 2-propanol, 1-heptanol, 1-hexanol, 1-butanol, 1-propanol, ethanol, and methanol.

In one embodiment, the interphase layer is collected in the above-mentioned step 4). In one embodiment, the method further comprises obtaining a single-molecule product from the interphase layer that comprises a mixture of different molecules that are unable to go in the non-polar or water layer. According to the subject invention, obtaining a single-molecule product from the interphase layer comprises obtaining an evaporated product from the interphase layer by, for example, using a rotary evaporator, mixing the evaporated product of the interphase layer with a non-polar solvent such as chloroform, centrifuging the mixture, for example, at 10000 g for 30 min, and collecting the single-molecule product.

In some embodiments, one or more microbial metabolites are present in the interphase layer. The interphase layer can be treated as the non-polar layer according to the subject invention. The above mentioned step for extracting single-molecule products from the non-polar layer can be repeated by mixing the evaporated product with different solvents with increased polarity until each of the microbial metabolites is extracted. The solvents may be used in an order of, for example, chloroform, acetone, 2-butanol, 2-propanol, 1-heptanol, 1-hexanol, 1-butanol, 1-propanol, ethanol, and methanol.

In one embodiment, the cell layer is collected in the above-mentioned step 4). In one embodiment, the method according the subject invention further comprises obtaining a single-molecule product from the cell layer. The cell layer comprises cells and proteins. The cell layer can be treated similarly to the water layer according to the subjection invention. The above-mentioned step for extracting single-molecule products from the water layer can be repeated by mixing the evaporated product with different solvents with increased non-polarity until each of the microbial metabolites is extracted. The solvents may be used in an order of, for example, methanol, ethanol, 1-propanol, 1-butanol, 1-hexanol, 1-heptanol, 2-propanol, 2-butanol, acetone, and chloroform.

In certain embodiments, the methods of the subject invention can be carried out in such a way that minimal-to-zero waste products are produced, thereby reducing the amount of fermentation waste being drained into sewage and wastewater systems, and/or being disposed of in landfills. Furthermore, this can be achieved while increasing the overall microbial metabolite production from a single fermentation cycle.

The cell biomass collected from the culture after removal and purification of the microbial metabolites would typically be inactivated and disposed of. However, the subject methods can further comprise collecting the cell biomass and using it, in live or inactive form, for a variety of purposes, including but not limited to, as a soil amendment, a livestock feed supplement, an oil well treatment, and/or a skincare product. The cell biomass can be used directly, or it can be mixed with additives specific for the intended use.

Combined with the characteristics of low toxicity and biodegradability, microbial metabolites such as SLP are advantageous for use in many settings including, for example, improved bioremediation, mining, and oil and gas production; waste disposal and treatment; enhanced health of livestock and other animals; food additives, such as preservatives and/or emulsifiers; cosmetic additives; and enhanced health and productivity of plants.

In one embodiment, the subject invention further provides methods for producing a composition comprising one or more purified microbial metabolites. In a specific embodiment, the subject invention provides methods for producing a purified sophorolipid (SLP) composition.

In certain embodiments, the subject invention provides compositions produced according to the subject methods, the compositions comprising a purified microbial metabolite, for example, SLP, and water. In preferred embodiments, the percentage of water in the composition, by volume, is about 1% to 50%, about 5% to 50%, about 10% to 40%, preferably about 20% to 30%.

In one embodiment, the purified SLP of the composition comprises purified hydrophilic SLP. In a specific embodiment, the composition comprises SLP with a purity of at least 95%, 96%, 97%, 98%, 99%, or 99.9%.

In a specific embodiment, the purified SLP of the composition can be, for example, a linear, mono-acetylated linear, and/or di-acetylated linear sophorolipid. In certain embodiments, the composition comprises more than one purified SLP molecule. In one embodiment, the combined composition may be achieved by combining each purified SLP together.

In some embodiments, the composition can be stored in a container until use. The storage time is preferably short. Thus, the storage time may be less than 60 days, 45 days, 30 days, 20 days, 15 days, 10 days, 7 days, 5 days, 3 days, 2 days, 1 day, or 12 hours.

Further components can be added to the composition according to the subject invention, e.g., sophorolipidic compositions, as needed for a particular use. The additives can be, for example, buffers, carriers, other microbe-based compositions produced at the same or different facility, viscosity modifiers, preservatives, nutrients for microbe growth, nutrients for plant growth, solvents, tracking agents, pesticides, herbicides, animal feed, food products and other ingredients specific for an intended use.

In one embodiment, the composition can be used as an active ingredient and/or as an adjuvant in a household or industrial cleaning composition or detergent, such as, e.g., a surface cleaner or a dish or laundry detergent.

In one embodiment, the composition can be used as an active ingredient and/or as an adjuvant in a cosmetic composition, such as, e.g., lotions, creams, shampoos, masks, gels, make-up or ointments.

In one embodiment, the composition can be used as an active ingredient and/or as an adjuvant in an agricultural composition, such as, e.g., a soil amendment, a pesticide, an herbicide, a biostimulant, a foliar treatment or a fertilizer.

In one embodiment, the composition can be used as an active ingredient and/or as an adjuvant in an oil and gas industry composition, such as, e.g., a completion fluid, a drilling fluid, a fracking fluid, a cleaning treatment, a wax dispersant or inhibitor, an acidizing fluid, a viscosity modifier, an emulsifier or de-emulsifier.

In one embodiment, the composition can be used as an active ingredient and/or as an adjuvant in a health-promoting, nutritional and/or therapeutic composition for animals and/or humans, such as, e.g., a pharmaceutical, a supplement, a hydration enhancer, a food or beverage, or a probiotic composition.

Cultivation of microbial biosurfactants, polyketide, and/or other metabolites according to the prior art is a complex, time and resource consuming, process that requires multiple stages. Advantageously, the methods of the subject invention do not require complicated equipment or high energy consumption, and thus reduce the capital and labor costs of producing microorganisms and their metabolites on a large scale. Additionally, the methods and equipment of the subject invention reduce the capital and labor costs of purifying microbial metabolites on a large scale.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Cultivation of Starmerella bombicola for Linear SLP Production and Purification

### Preparation

A stainless steel fermentation reactor is used for the production of SLP. The reactor comprises about 150 gallons of water, into which a medium comprising dextrose (25 to 150 g/L), yeast extract (1 to 10 g/L), canola oil (25 ml/L to 110 ml/L) and urea (0.5 to 5 g/L) is added.

The reactor comprises a mixing apparatus for continuous agitation and mixing of the culture. The reactor with medium is steamed at 100° C. for about 60 minutes in order to sterilize the reactor and the growth medium.

The reactor is then allowed to cool down. Once the reactor reaches about 35° C., antibiotics are added to the medium to prevent bacterial contamination. The antibiotic composition comprises 300 g streptomycin and 20 g oxytetracycline dissolved in 4 L DI water. Other reactor tubing and openings are sprayed with isopropyl alcohol (IPA) to sterilize them.

Small-scale reactors are used for growing Starmerella bombicola inoculum cultures. The culture is grown for at least 42 to 48 hours at 26 to 28° C. in the small-scale reactors. Once the stainless-steel fermentation reactor reaches 30° C., it is then inoculated with about 25 L of the inoculum culture.

### Fermentation

The temperature of fermentation is held at 23 to 28° C. After about 22 to 26 hours, the pH of the culture is set to about 3.0 to 4.0, or about 3.5, using 20% NaOH. The fermentation reactor comprises a computer that monitors the pH and controls the pump used to administer the base, so that the pH remains at 3.5. After about 6-7 days of cultivation (120 hours+/−1 hour), the batch is ready for harvesting.

### Extraction and Purification

The culture broth is centrifuged to remove lactonic SLP and cells, and is mixed with ethyl acetate at a 1:1 ratio for at least 24 h. The mixture is then centrifuged at 10000 g for 30 min. The non-polar layer, interphase layer, and water phase layer that form are collected separately.

The non-polar and water layers are each centrifuged again at 10000 g for 30 minutes. The supernatant of each of the non-polar and water layers is collected separately and evaporated using a rotary evaporator. The temperature for the rotary evaporator is preferably no higher than 45° C. in order not to cause product degradation.

The evaporated product of the water layer contains only linear monoacetylated SLP with oleic acid tail and some residual caramelized glucose. This product is mixed with 100% methanol because glucose is mostly insoluble in methanol. Such reaction can be carried out at room temperature, or for higher yields, at lower temperatures. The solution is mixed for 24 hours before being centrifuged at 10000 g for 30 min. After centrifugation, the glucose precipitates and is discarded. The methanol layer is collected and evaporated using the rotary evaporator. The evaporated product contains a single molecule product, i.e., linear monoacetylated SLP.

### Example 2—Production and Purification of Isopropyl Fatty Acid Esters

Meyerozyma guilliermondii are used to produce fatty acid esters. A fermentation reactor with 150 gallons of sterilized, deionized water and a growth medium is inoculated with M guilliermondii yeast cells to produce a yeast culture. The growth medium comprises:

| Ingredient | Concentration | |
|---|---|---|
| Ammonium Nitrate | 10 | g/L |
| Potassium Phosphate Dibasic | 2.5 | g/L |
| Sodium Phosphate monobasic | 0.15 | g/L |
| Magnesium Sulfate | 0.5 | g/L |
| Calcium Chloride | 0.1 | g/L |
| Manganese Sulfate | 0.02 | g/L |
| Casein Peptone | 1 | g/L |
| Dextrose | 5 | g/L |
| Soybean Oil | 10 | ml/L |

A 4 L solution with 300 g of streptomycin and 20 g of oxytetracycline is added to the fermentation reactor. Before the initial 22-24 hours of fermentation, the pH is adjusted to 5.5 using an aqueous HCl solution. The temperature is initiated and then maintained at 24° C. and the dO₂ remains at about 35% to about 65% during fermentation.

After the initial 22-26 hours of fermentation, an aqueous base solution comprising 20% NaOH is fed into the reactor to adjust and maintain pH automatically at about 6.95 to about 7.05. Sampling for the amount of soybean oil is performed every day during fermentation. Foam containing some fatty acid esters is collected continuously throughout the fermentation process.

Total fermentation time is about 94 to about 98 hours. After the cycle ends, the yeast culture is placed into a tank and allowed to settle for up to 24 hours. An upper layer containing fatty acid esters forms and is separated from a lower, aqueous layer.

Isopropyl alcohol is added to the fatty acid ester layer and mixed in a collection vessel for about 1 hour. After the vessel settles for about 24 hours, a top fatty acid ester layer and a bottom layer forms. The bottom layer is removed and discarded. Any foam that had been collected during fermentation is mixed with the remaining fatty acid ester layer to produce a crude isopropyl ester solution.

The crude isopropyl ester solution can contain isopropyl oleic/linoleic/palmitic acid esters as well as some free fatty acid esters and residual oleic/linoleic/palmitic acid. This crude solution is mixed with 10% hexanol at 1:1 ratio for 24 h. The mixture is centrifuged at 10000 g for 30 min. Pure isopropyl palmitic acid esters are extracted into the hexanol phase because they are more polar than all other compounds in the solution. A product that contains isopropyl palmitic acid ester (up to 98% purity) is obtained by evaporating the hexanol.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

1. A method for extracting one or more microbial metabolites at a high level of purity, the method comprising:
1) cultivating a metabolite-producing microorganism to produce a microorganism culture broth, said microorganism culture broth comprising liquid fermentation medium, microorganism cells and one or more microbial metabolites;
2) mixing the culture broth with a non-polar solvent;
3) centrifuging the mixture of step 2) to cause phase separation in the mixture, wherein a non-polar layer, an interphase layer, a water layer and a cell layer are separated;
4) separately collecting one or more layers selected from the non-polar layer, interphase layer, water layer and cell layer;
5) independently centrifuging one or more collected layers of step 4) and independently collecting one or more supernatants from one or more collected layers; and
6) obtaining one or more microbial metabolites by evaporation.

2. The method of claim 1, wherein the metabolite-producing microorganism is a yeast.

3. The method of claim 2, wherein the yeast is Starmerella bombicola or Meyerozyma guilliermondii.

4. The method of claim 1, wherein the non-polar solvent is ethyl acetate.

5. The method of claim 1, wherein the culture broth and the non-polar solvent is mixed at a ratio of from 1:10 to 10:1.

6. The method of claim 5, wherein the culture broth and the non-polar solvent is mixed at a ratio of 1:1.

7. The method of claim 1, wherein the high level of purity is at least 95%.

8. The method of claim 1, wherein the microbial metabolite is a sophorolipid (SLP).

9. The method of claim 8, wherein the SLP is hydrophilic SLP comprising di-acetylated linear SLP and/or mono-acetylated linear SLP.

10. The method of claim 1, wherein the water layer is collected in step 4).

11. The method of claim 10, wherein the method further comprising isolating one or more microbial metabolites from the water layer, which comprises:
i) obtaining an evaporated product of the water layer;
ii) mixing the evaporated product of the water layer with an alcohol;
iii) centrifuging the mixture of step ii) and collecting the alcohol layer; and
iv) obtaining the microbial metabolite by evaporating the alcohol.

12. The method of claim 1, wherein the alcohol is methanol or ethanol.

13. The method of claim 1, wherein the non-polar layer is collected in step 4).

14. The method of claim 13, wherein the method further comprising purifying one or more microbial metabolites from the non-polar layer, by:
i) obtaining an evaporated product of the non-polar layer;
ii) mixing the evaporated product of the non-polar layer with chloroform;
iii) centrifuging the mixture of step ii) and collecting the top layer; and
iv) obtaining the microbial metabolite by evaporation.

15. The method of claim 10, wherein the method further comprising purifying each of one or more microbial metabolites from the water layer, which comprises a plurality of purifying cycles using solvents with increased non-polarity, each cycle comprising:
i) obtaining an evaporated product of the water layer;
ii) mixing the evaporated product of the water layer with a solvent;
iii) centrifuging the mixture from step ii) and collecting the solvent layer; and
iv) obtaining a microbial metabolite by evaporating the solvent;
wherein each cycle uses a different solvent that is more non-polar than the solvent used in the previous cycle.

16. The method of claim 15, wherein the solvents in the plurality of purifying cycles are used in an order of: methanol, ethanol, 1-propanol, 1-butanol, 1-hexanol, 1-heptanol, 2-propanol, 2-butanol, acetone, and chloroform.

17. The method of claim 13, wherein the method further comprises purifying each of the one or more microbial metabolites from the non-polar layer, which comprises a plurality of purifying cycles using solvents with increased polarity, each cycle comprising:
i) obtaining an evaporated product of the non-polar layer;
ii) mixing the evaporated product of the non-polar layer with a solvent;
iii) centrifuging the mixture from step ii) and collecting the solvent layer; and
iv) obtaining a microbial metabolite by evaporating the solvent;
wherein each cycle uses a different solvent that is more polar than the solvent used in the previous cycle.

18. The method of claim 17, wherein the solvents in the plurality of purifying cycles are used in an order of: chloroform, acetone, 2-butanol, 2-propanol, 1-heptanol, 1-hexanol, 1-butanol, 1-propanol, ethanol, and methanol.

19. A composition produced by the method of claim 1, the composition comprising a purified SLP and a percentage of water, wherein the percentage of water is less than 50%, and the SLP has a purity of at least 95%.

20. The composition of claim 19, wherein the percentage of water is 20% to 30%.

21-26. (canceled)
